(19) United States
(12) Patent Application Publication
(10) Pub. No.: US 2011/0203946 A1
(43) Pub. Date: Aug. 25, 2011

(54) Formulation

(76) Inventors: Edward McCloskey, Louth (IE); Gemma Seery, Dublin (IE); John Woodward, Meath (IE); Ronald Russell, Dublin (IE)

(21) Appl. No.: 12/996,171

(22) PCT Filed: Jun. 4, 2009
(86) PCT No.: PCT/EP09/56890

§ 371 Date: Dec. 31, 2010

(30) Foreign Application Priority Data
Jun. 4, 2008 (IE) S20080450

(51) Int. Cl. B65D 81/24; A01N 59/00; A61K 8/02; A61K 8/67; B65D 85/00

(52) U.S. Cl. 206/205; 424/661; 424/401; 424/53; 206/524.1

(57) ABSTRACT

This invention relates to a formulation for use in personal care products, cleaning products and the like. Ideally, the formulation is a natural preservative and disinfectant formulation comprising activated water and grapefruit seed extract.

### CROSS REFERENCE TO RELATED APPLICATIONS

The present application claims priority to PCT Application No. PCT/EP2009/056890 filed 4 Jun. 2009, which in turn claims priority to Irish Patent Application No. S2008/0450 filed 4 Jun. 2008, said applications being incorporated in their entirety herein by reference thereto.

### FEDERALLY SPONSORED RESEARCH AND DEVELOPMENT

None.

### BACKGROUND OF THE INVENTION

1. Field of the Invention

The present invention relates to a natural preservative and disinfectant formulation for use in personal care products, cleaning products and the like and for use as an antimicrobial agent.

2. Background

Personal care products such as hygiene, cosmetic, baby, oral, skin and hair care products (including, for example, wet wipes, creams, lotions, deodorants, formulations, sprays, powders, make-up, shampoo, mousse, paste, gels etc) typically include formulations containing chemical preservatives. The same is true of various cleaning products, whether they are baby cleaning wipes, cosmetic cleaning wipes or surface cleaning wipes generally for use in the kitchen or bathroom for example.

Such preservative agents are necessary to protect the product prior to use from growing mould, yeast or bacteria etc and, thus, are important from a hygiene point of view and also in extending the shelf-life of such products. For example, in the routine manufacture of wet wipes, an industrial preservative such as Nipagard® BPX', containing chemical biocides, prevents the spoilage of wipes. However, such chemical preservatives can be viewed as harmful to people and to the environment, and are often an irritant to those with sensitive skin.

Thus, the use of such industrial and chemical additives/biocides to prevent spoilage in personal care products, cleaning products and the like is undesirable from both a consumer and environmental perspective. This is because they can cause skin irritations, and because microbes can build up resistance to them. In addition, there is growing interest in the baby care market especially for products which are as pure and mild and chemicals-free as possible. This is particularly important because of the risk of skin sensitisation for babies and children.

Thus, there is a need to find alternative spoilage-inhibitory treatments which do not contain such conventional undesirable chemical additives yet are compatible with the products and their end uses.

It is an object of the present invention to overcome these problems.

### SUMMARY OF THE INVENTION

According to a general aspect of the invention, there is provided a formulation incorporating activated water as an ingredient. The invention envisages the use of activated water only; a formulation which includes activated water; and other natural additional ingredients. The invention also envisages other formulations which include activated water in which the use of activated water helps to reduce, minimise or eliminate the quantities of conventional disinfectant and/or preservative agents, such as chemical agents for example, used in the formulations.

According to a first aspect of the invention, there is provided a natural disinfectant and preservative formulation comprising electrochemically activated water and grapefruit seed extract.

The formulation as defined above may be used in the manufacture of personal care products and cleaning products, such as those mentioned above, wherein the activated water in the formulation acts as a disinfectant agent and/or a preservative agent. Thus, ideally the activated water will initially disinfect the product itself (whether personal care or cleaning product) and/or the interior of the packaging for the product where provided, and thereafter the grapefruit seed extract acts as a form of preservative over a period of time.

According to a second aspect of the invention, there is provided a personal care product comprising the natural disinfectant and preservative formulation according to the invention.

According to a third aspect of the invention, there is provided a wet wipe comprising a substrate impregnated with natural disinfectant and preservative formulation, preferably comprising at least 50 ppm activated water and at least 500 ppm grapefruit seed extract.

According to a fourth aspect of the invention, there is provided a method of disinfecting and preserving a personal care product or medical device and associated packaging comprising the use of the formulation of the invention.

According to a fifth aspect of the invention, there is provided the use of the formulation of the invention in therapy as an antimicrobial agent.

### BRIEF DESCRIPTION OF THE DRAWINGS

The invention will be more clearly understood by the following description of some embodiments thereof, given by way of example only, with reference to the accompanying drawings.

FIG. 1 is a perspective view of a wet wipe pack according to the invention.

FIG. 2 is a sectional view of the wet wipe pack of FIG. 1 taken along the line 11-11 of FIG. 1.

FIG. 3 is a perspective view of another wet wipe pack according to a second embodiment of the invention.

FIG. 4 shows a comparative log reduction for bacterial and fungal species exposed to 20 ppm electrochemically activated water for 10 and 20 minutes respectively.

### DETAILED DESCRIPTION OF THE INVENTION

In the specification, it will be understood that the terms “antimicrobial” “antibacterial” or “microbicidal” are used interchangeably herein and cover biocidal or biostatic activity against various types of micro-organisms including but not limited to bacteria, fungi, viruses, yeasts, parasitic or pathogenic micro-organisms and/or moulds. The disinfectant effect of the invention is essentially an anti-microbial effect.

In this specification, the term “natural disinfectant and preservative formulation” will be understood to cover a formulation with antimicrobial activity. The antimicrobial activity involves the restriction of growth and/or killing of the micro-organisms. Such a formulation is termed a “natural” formulation as it does not comprise any significant levels (i.e. not present or below typical trace levels) of conventional biocides/pesticides or allergans present in conventional disinfectant/preservative formulations.

In this specification, the terms “electrochemically activated water” or “electrochemically activated solution” or “hydroactive water” will be understood to be interchangeable and relate to water that has undergone electrochemical activation (ECA) yielding peroxy compounds with antimicrobial characteristics. ECA treatment involves the exposure of water with natural salts (e.g. sodium chloride, sodium carbonate, sodium bicarbonate, potassium carbonate, potassium bicarbonate and/or calcium carbonate) added to it, to a substantial electrical potential difference. If an anode (+) and a cathode (−) is placed in pure water and one applies a direct current, electrolysis of the water will occur at the poles leading to a breakdown of the water into its constituent elements, producing gaseous hydrogen and oxygen. However, if sodium chloride is added to the water to form a solution, the dominant electrolysis end product is hypochlorite or hypochlorous acid (HOCl), a chlorine-based reagent which may be used to kill microorganisms. It will be understood that electrochemically activated water contains metastable hypochlorous acid and its mixed oxidant intermediates and is an effective disinfectant against all forms of microbes including spores and protozoa. Such solutions are initially highly activated and toxic to single-celled organisms, including bacteria, fungi, yeasts and spores, through both chemical and physical effects. This activity is measured by redox potential and hypochlorous acid (free available chlorine (FAC)). Within approximately a period of hours to days (eg 4 to 48 hours) depending on storage and other factors, this activity declines and the solution reverts to its starting materials, common salt and water.

In this specification, the term “grapefruit seed extract” (GSE) relates to an extract of grapefruit seeds, ideally derived from only the seeds of the grapefruit, Citrus grandis, (Rutaceae). GSE is a widely commercially available product formed by extracting the seeds of grapefruit with propylene glycol and/or glycerine. Some commercially available GSEs are made from a combination of one or more of seed, pulp, juice, leaf, oil, peel, glycerin and/or synthetic preservatives all blended together. In this specification, the GSE ideally comprises a specifically targeted extract of grapefruit seeds only, ideally without any contamination from other parts of the grapefruit i.e. pulp, juice, leaf etc.

In addition, a conventional GSE (often a waste stream derivative) may be contaminated by one or more of the following: quaternary ammonium compounds such as benzethonium chloride and/or benzalkonium chloride; Triclosan, a polychloro phenoxy phenol; conventional pesticides/biocides; and/or allergans.

Indeed, the pesticide/biocide derived contaminants have been found to be possibly responsible for much of the biocidal effect of conventional GSE. Thus, the GSE of the invention is pure in the sense it is not contaminated with or has only trace contaminants of the above contaminants. Accordingly, such contaminants are ideally not present or present at or below typical trace levels, preferably less than 2 mg/kg of GSE.

In addition, Grapefruit Seed Extract itself contains bioflavonoids, natural polyphenolic substances including quercitin, rutin, naringin, isonaringin and hesperidin. It will be understood that some of these polyphenols may be chemically converted during GSE processing into more stable substances such as diphenol hydroxybenzene (a quaternary ammonium compound) and these specific quaternary ammonium compounds may be present in the GSE above, at or below trace levels as defined above. Thus, quaternary ammonium compounds, but excluding diphenol hydroxybenzene, may be present at or below trace levels of the GSE of the invention.

It will be understood that the GSE of the invention may be provided with additional components such as glycerine and Vitamin C (ascorbic acid).

In the specification the term “by weight”, “percentage by weight” or “w/w %” refers to the weight of the final composition or system. In the specification the term “by volume”, “percentage by volume” or “v/v %” refers to the volume of the final composition or system. These values are interchangeable with w/v.

It will be understood that where parts per million (ppm) are referred to for ECA solutions, ppm refers to the free available chlorine (FAC) measurable or detectable in the ECA solution. It is known that ECA solutions can be generated at different strengths of free available chlorine (FAC). Thus, for example an ECA solution generated at 200 ppm must be diluted to 100 ppm using a diluent such as water (e.g. purified, sterilized or filtered water), typically reverse osmosis (RO) water. Thus, in this situation the percentage by volume (v/v) is approximately 50% ECA solution based on total water volume. Hence, the dilution factor of the ECA will vary depending on the strength of the ECA at time of manufacture. For GSE, ppm refers to its volumetric content within the diluent i.e. water content.

According to the main aspect of the invention, there is provided a natural disinfectant and preservative formulation comprising electrochemically activated water and grapefruit seed extract.

The formulation of the invention may be dispersed in filtered, sterilised or purified water, such as reverse osmosis water. Alternatively, the formulation of the invention may be supplied as an additive for combination (e.g. dispersion/impregnation/addition) with a substrate (whether liquid, gel or solid substrate).

Preferably, the formulation of the invention comprises at least 1 ppm, preferably 20 ppm, more preferably 50 ppm or greater, even more preferably 100 ppm or greater, electrochemically activated water (ECA) and at least 500 ppm, preferably 1000 ppm or greater, grapefruit seed extract.

Levels above these values may be contemplated for both the GSE and ECA. Higher levels of GSE and ECA may be contemplated depending on the end use of the formulation and the microbial load/bio-burden of the substrate the formulation is being applied to or combined with. For example, 150 ppm, 200 ppm, 250 ppm, 1000 ppm, 1200 ppm or greater of ECA may be used. Alternatively, 1200 ppm, 1500 ppm, 2000 ppm, 2500 ppm or greater of GSE may be used.

Advantageously, and unexpectedly, the combination of the electrochemically activated water and the grapefruit seed extract in the formulation of the invention provides for an efficacy, in terms of disinfection and/or preservative effect, greater than the efficacy of either administered alone. This exponential efficacy of this specific combination is unexpected.

Optionally, the electrochemically activated water in the formulation has one or more of the following properties; a pH of from 2 to 9, preferably 3 to 8.5, more preferably from 6.5 to 7; a redox potential of greater than 400 mV, preferably greater than 600 mV; and/or from 1 to 150 ppm free available chlorine (FAC), preferably from 5 to 100 ppm free available chlorine (FAC). Higher levels are also contemplated.

The formulation may also comprise electrochemically activated water comprising greater than 50%, preferably greater than 75% anolyte.

Optionally, the grapefruit seed extract in the formulation has one or more of the following properties:
the GSE is pure and not contaminated with or has only trace contaminants of pesticide and/or synthetic biocide derived contaminants and/or bioflavonoid derived contaminants present in many known GSEs, including quaternary ammonium compounds and benzalkonium chloride. Ideally, such contaminants are found at a level of equal to or less than 2 mg/kg (“trace levels”) of GSE. Preferably, such contaminants are present at a level less than 0.1% v/v impurities.
the GSE may be provided with additional components such as glycerine and Vitamin C (ascorbic acid).
general anti-microbial properties in killing and/or arresting the growth of micro-organsims—bactericidal against gram positive and gram negative bacteria and prevents growth of many yeasts such as Candida albicans, Malaseezia globosa and Aspergillus nidulans.
comprises approximately 40 to 50%, ideally 45% v/v dextrose free grapefruit seed extract; 30 to 40%, ideally 34% v/v glycerine; and 15 to 25%, ideally 18% v/v vitamin C (ascorbic acid).

Ideally, the formulation of the invention comprises grapefruit seed extract present at a level 500 ppm or greater.

According to preferred embodiment of the invention, the natural disinfectant and preservative formulation comprises electrochemically activated water at a level of 100 ppm or greater and GSE at a level of 1000 ppm or greater. We have found that levels of GSE of 1000 ppm or greater and ECA of 100 ppm or greater are particularly advantageous and provide for a synergistic effect.

Although, it will be understood that in some embodiments the formulation may comprise GSE and ECA only, from a practical perspective, it is preferred to make the GSE and ECA as concentrates and dose these into the diluent, preferably water, more preferably reverse osmosis water, to achieve the desired concentration.

For example, in a preferred embodiment of the invention, the formulation comprises 100 ppm ECA and 1000 ppm GSE and a diluent, such as purified, sterilized or filtered water. Ideally, the diluent is reverse osmosis (RO) water. Thus, in this preferred embodiment, GSE is present in the formulation at a concentration of approximately 0.1% v/v based on the diluent. Ideally, the ECA solution is present in the formulation at a concentration of 0.01% v/v FAC based on the diluent. Hence, the diluent, whether purified, sterilized or filtered water, makes up the remainder of the formulation.

In other embodiments of the invention, other levels (whether higher or lower) of GSE and ECA are contemplated.

In another embodiment, the formulation comprises approximately 95% to approximately 99.9% by weight water (ECA and diluent) and from approximately 0.1% to approximately 5% by weight grapefruit seed extract based on the weight of total formulation. Ideally, the formulation comprises approximately 99.9% by weight water and 0.1% by weight grapefruit seed extract based on the weight of total formulation.

It will be understood that the natural disinfectant and preservative formulation of the invention may be in the form of a solid, liquid or gel or may be combined with or applied to a solid, liquid or gel substrate or matrix.

For example, according to one embodiment the formulation may be present within a solid matrix or substrate such as a non-woven (e.g. cotton) wipe or viscose or polypropylene-based interleaved sheet. In this manner the formulation of the invention is impregnated in the solid substrate.

Alternatively, according to another embodiment the matrix may be provided in combination with, e.g. dispersed within, a liquid or gel matrix, such as an emulsion or emollient composition.

Additionally, the formulation may be provided as a liquid spray. In this form, it will be understood that additional excipients may not necessarily be required as the formulation is already in liquid form (with the purified/sterilised water acting as the carrier). However, optional excipients may be added, for example to add fragrance, alter the viscosity of the liquid etc.

It may also be provided in the form of liquid gel tablets or capsules, for later delivery or introduction by squeezing/rupturing. Additional excipients may be required to provide a gel-like matrix for the formulation of the invention or the formulation may simply be encased in a gel tablet casing.

Preferably, the formulation of the invention is used in a personal care product. Such personal care products include but are not limited to wet wipes, creams, lotions, deodorants, formulations, sprays, powders, make-up, shampoo, mousse, paste and/or gels.

We have unexpectedly found that the formulation of the invention comprising a combination of electrochemically activated water at 20 ppm, preferably 50 ppm, more preferably 100 ppm or greater and GSE at 500 ppm or greater, preferably 1000 ppm or greater results in a shelf-stable contaminant-free formulation. It will be understood that although the GSE is contaminant-free, trace levels of contaminants may be present. Surprisingly, we have found that such a formulation provides a similar or better disinfectant and preservative activity as the combination of regular pure water and a conventional chemical preservative, eg Nipaguard®. Thus the activity of the formulation of the invention matches the activity of conventional chemical preservative formulations.

It will be understood that GSE of the invention may be provided with additional components such as glycerine and Vitamin C (ascorbic acid), however, contaminants (quaternary ammonium compounds/pesticides/allergens and/or triclosan) will either not be present or be present at or below typical trace levels.

Ideally, the formulation is used to initially disinfect the matrix or substrate it is applied to, whether solid, liquid or gel type substrate/matrix. Additionally, the formulation is also used to disinfect the packaging for the matrix/product it is applied to.

Ideally, the formulation is present within a sealed container. In a particularly preferred embodiment the activated water or formulation including activated water is applied to or incorporated in a product contained within a sealed package or container. Accordingly, it may also be used to disinfect the packaging of the sealed container.

Subsequently, it may be used as a preservative within the formulation to prevent any further antimicrobial growth.

When used in a personal care product, such as a wet wipe (solid matrix) or a cosmetic composition (liquid or gel matrix), the multifunctional and synergistic actions of the electrochemically activated water and GSE in the formulation combine to repair and sooth irritated, broken or damaged skin and improve skin integrity and hydration. In addition, the combined antioxidant effects may reduce the sensitization potential to the formulation.

Advantageously, the formulation of the invention does not result in irritation or sensitisation.

We also postulate that the combined effects of GSE and the electrochemically activated water enhance the recovery of damaged skin in occluded areas. GSE itself and the glycerine would help maintain good skin condition and reduce any inflammatory response and Vitamin C would be rapidly absorbed through the epidermis to repair the underlying tissues.

Advantageously, the activated water can be used as a substitute, either partially or fully, for chemical preservatives which are conventionally used with such products. Thus, the formulation of the invention is non-toxic and non-irritant.

Furthermore, the formulation of the invention may protect the product in both closed and open conditions, e.g. a sealed pack of wet wipes once opened will still be protected by the formulation.

The formulation of the invention will now be described in relation to different embodiments it may be used with.

A preferred embodiment is a wet wipe comprising the formulation of the invention. In this aspect of the invention there is provided a wet wipe comprising a substrate or matrix (these terms are interchangeable) impregnated with the formulation of the invention. Thus, it will be appreciated that the invention proposes providing a wet wipe simply impregnated with the formulation of the invention and without the conventional anti-microbial/anti-bacterial agents, preservatives and the like.

According to a preferred embodiment, there is provided a wet wipe comprising a substrate impregnated with the natural disinfectant and preservative formulation of the invention, comprising at least 50 ppm, preferably 100 ppm activated water and at least 500 ppm, preferably 1000 ppm, grapefruit seed extract.

Preferably, the wet wipe of the invention comprises a wet wipe impregnated with a formulation comprising approximately 95% to approximately 99.9% by weight water (ECA and diluent) and from approximately 0.1% to approximately 5% by weight grapefruit seed extract based on the weight of total formulation. Ideally, an ultra-pure and mild wet wipe is impregnated with a formulation comprising approximately 99.9% by weight water and 0.1% by weight grapefruit seed extract based on the weight of total formulation.

In another aspect the invention provides a wet wipe pack comprising a sealed outer package containing one or more wet wipes, each of said wet wipes comprising a substrate impregnated with the formulation of the invention. When sealed within the package, the activated water acts as an anti-microbial/anti-bacterial agent and preservative.

In one embodiment a single wet wipe is contained within the package.

In another embodiment a plurality of wet wipes are contained within the package. In this arrangement conveniently a stack of wet wipes may be provided in the package. These may be interlinked by tear-away perforations. Alternatively, the wipes could be interleaved.

Conventional flow-wrap packaging, with a peel-and-reseal sticker over the opening, may be used to pack the wet wipes.

Where a plurality of wet wipes is provided in the package it may also be desirable that the package has an elastomeric dispensing opening. Such an arrangement is described for example in our previously filed European Patent Publication No. EP 1 483 171. This elastomeric opening has the added function of keeping the contents of the (opened) pack away from contact with any contaminants. Additionally this elastomeric opening may include actively anti-microbial agents such as nano silver particles and the like.

If desired also the package may be provided with a dispensing opening with a tear-open cover mounted across the dispensing opening. Again, such an arrangement is described in our earlier Patent Publication No. EP 1 483 171.

The substrate may comprise a wipe, cloth, pad or the like and may be of any suitable material and construction. Any suitable materials may be used, both natural and synthetic, either separately or in combination. Woven or non-woven materials may be used. Pad constructions comprising an outer envelope containing particulate filler material could be used also. Such woven and non-woven materials inherently contain contaminants and the formulation of the invention provides an initial anti-microbial effect against these inherent contaminants (both bacterial and fungal contaminants in varying amounts attached to or embedded in the matrix). The formulation then provides a disinfectant and preservative effect to give the product a long shelf life, greater than 12 months, preferably 24 months, more preferably 36 months.

According to another embodiment of this aspect of the invention, there is provided a wet wipe pack comprising a sealed outer package containing one or more wet wipes, wherein each of said wet wipes comprising a substrate impregnated with the natural disinfectant and preservative formulation of the invention.

Ideally, the substrate is a wipe in the form of a stack of interleaved sheets. The sealed container is a plastic pouch or a plastic envelope with elastomeric opening as described in EP 1 483 171.

We postulate that the electrochemically activated water naturally disinfects the substrate during manufacture. The wet wipe is packaged in a closed container or plastic pouch, thereby maintaining the sterility of the wet wipe until opening. Once opened, we have surprisingly found that the synergistic interaction of the GSE and electrochemically activated water results in a wet wipe with extended shelf life.

It will be appreciated that the activated water initially disinfects the product (e.g. a bundle of wipes), the formulation/water and the interior of the packaging. Depending on the manufacturing environment, the strength of the activated water used and the bacterial loading disinfected by the activated water, the activated water maintains residual preservative properties for a period of time. To enhance this residual preservative effect in the product, in the manufacturing process a double dosing of activated water may be used. Thus, a first dosing of activated water prepares the product by disinfecting it and a subsequent second dosing of activated water enhances the preservative properties.

According a preferred embodiment of the invention, to provide a disinfection effect outside of the wet wipe itself and a capability beyond that of providing a long shelf-life only, ideally, electrochemically activated water at greater than 200 ppm and GSE greater than 2000 ppm may be used.

When such a wet wipe is being manufactured, ideally, a bundle of wipes are soaked in an excess of the formulation of the invention. The wetted bundle of wipes are then packed under conventional aseptic conditions. Alternatively, the formulation may be applied to a single wipe which is then aggregated to form a bundle of wet wipes and packaged under conventional aseptic conditions. Still optionally, the activated water may be applied to the wipes initially, followed by the GSE, and packaged under conventional aseptic conditions.

### Grapefruit Seed Extract

Grapefruit Seed Extract has been used for many years in oral and topical herbal and alternative medicines for its antiseptic and antibacterial properties at concentrations higher than 0.5%. It is also used as a broad spectrum disinfectant and decontaminant for food both for human and animal consumption at concentrations from 0.005-0.1% as a non-toxic, non-hazardous, non-corrosive, non-tainting, hypoallergenic and environmentally friendly disinfectant.

The water-soluble Citrus grandis grapefruit seed extract (GSE) in this formulation is similar in physico-chemical characteristics to those used in medicinal products but ideally at a lower concentration of approximately +/−0.1%.

According to another embodiment of the invention, the grapefruit seed extract is present in the range of 500 to 2500 ppm, ideally at a level 500 ppm or greater, more preferably 1000 ppm or greater. We have found that levels around 1000 ppm are particularly advantageous.

Optionally, the GSE may be present at levels of 1500 ppm or greater, preferably greater than 2000 ppm. Higher levels of GSE may be required if additional agents or formulation ingredients are used, for example, in some situations the material or substrate may have a high bio-burden and accordingly require a higher level of GSE.

According to a preferred embodiment of the invention, the formulation comprises approximately 100 ppm (0.01%) activated water and approximately 1000 ppm (0.1%), GSE. The remaining being purified, filtered or sterilised water, such as reverse osmosis water. This formulation is ideal for use with wet wipes.

According to an embodiment of the invention, the GSE of the invention is anti-microbial/anti-bactericidal against a panel of gram positive and gram negative bacteria, yeast, fungi and spores. Additionally, the GSE of the invention prevents growth of the yeast Candida albicans and the fungus Aspergillus nidulans at a dilution of approximately 1 in 2000 or 500 ppm in standard suspension tests as per EN 1040 and EN 1275.

Ideally, the GSE of the invention is pure and not contaminated with contaminants such as pesticides and/or biocides derived contaminants. Thus, trace levels (i.e. less than or equal to 2 mg/kg of GSE) are permitted. Accordingly, such contaminants including pesticide and/or biocide derived contaminants are found at a level of equal to or less than 2 mg/kg of GSE.

According to a preferred embodiment of the invention, the GSE comprises none or trace levels of quaternary ammonium compounds, triclosan, pesticides and/or allergans which are all commonly found in conventional GSE. Such trace levels are ideally at or below 2%, preferably at or below 0.1%, more preferably below 0.01%. Accordingly, such trace level pesticides (synthetic pesticides) would not have any efficacy as a preservative and would have no toxicological significance in the resultant GSE.

Pure GSE or dextrose free GSE may be used in the invention.

In addition, the GSE of the invention may be provided with additional components such as glycerine and Vitamin C (ascorbic acid).

Vitamin C is a powerful antioxidant. The antioxidant effects are enhanced in the presence of flavonoids. Vitamin C is a building block in collagen, an essential component of skin. Collagen fibres are ubiquitous throughout the body, providing firm but flexible structure for most tissues. In the skin, Vitamin C is involved in repair, maintenance and wound healing. It is readily absorbed systemically. It is also known that GSE naturally provides some of the Vitamin C, an antioxidant.

Glycerin, a humectant, reduces the acidity of the GSE and would also further decrease possible irritation to the skin or mucous membranes by urine or faeces. Its presence in the formulation reduces the acidity of GSE.

The following table outlines alternate names and functions of GSE and additional components.

| | | | | |
|---|---|---|---|---|
| INCI Name | Citrus grandis | Ascorbic acid | Glycerin | Aqua |
| | seed extract | (vitamin C) | | |
| INN Name | | ascorbic acid | glycerol | Water |
| Ph. Eur. Name | | acidium | glycerolum | aqua |
| | | ascoribicum | | |
| Chemical/ | Citrus Grandis | Ascorbic acid | Glycerol | Water |
| IUPAC Name | Seed Extract is | | | |
| | an extract of | | | |
| | the seeds of | | | |
| | the grapefruit, | | | |
| | Citrus grandis, | | | |
| | Rutaceae | | | |
| Functions | Astringent | Antioxidant | Denaturant | Solvent |
| | Skin | Buffering | Humectant | |
| | conditioning | Masking Skin | Perfuming | |
| | Tonic | Conditioning | Solvent | |

Grapefruit Seed Extract itself contains bioflavonoids, natural polyphenolic substances including quercitin, rutin, naringin, isonaringin and hesperidin. Flavonoids possess anti-oxidant and free radical scavenging activity and enhance the functionality of vitamin C by slowing down its metabolism. Flavonoids, as a class of chemicals, have the ability to modify the body's reaction to other compounds such as allergens. It will be understood that some of the polyphenols are chemically converted during the GSE processing into more stable substances such as diphenol hydroxybenzene (a quaternary ammonium compound) and these may be present in the GSE.

According to another embodiment of the invention, the grapefruit seed extract used in the formulation comprises approximately 35% to 50%, preferably 45% v/v dextrose free grapefruit seed extract, approximately 25% to 40%, preferably 34% v/v glycerine and approximately 15% to 25%, preferably 18% v/v vitamin C (ascorbic acid). GSE is hygroscopic so the remainder % v/v (approximately 1 to 10%, preferably 3%) comprises water.

### Activated Water

Water can be “activated” by applying an electric current across it. Preferably a small quantity of salt is added to the water to increase conductivity and the electric current is applied for a certain period of time. The water can be unequally separated into positive (anode) charged and negative (cathode) charged activated water. The positive charged activated water possesses a disinfectant ability.

According to one embodiment of the invention, a small quantity of the negative charged water may be mixed back with the positively charged water to balance the pH level and modify the properties to suit the application.

Thus, it will be understood that the electrochemically activated water (ECA) of the invention comprises anolyte and catholyte. Ideally, the electrochemically activated water comprises greater than 50%, preferably greater than 75% anolyte.

According to one embodiment the electrochemically activated water has a pH from 2 to 9, preferably 3 to 8.5, more preferably from 6.5 to 7.5, ideally 6.5.

According to another embodiment the electrochemically activated water has a redox value greater than 400 mV, preferably greater than 600-650 mV.

In another embodiment the activated water includes salt in solution.

According to a further embodiment, the electrochemically activated water contains metastable hypochlorous acid, ideally at concentrations between approximately 10 ppm and 200 ppm.

According to a further embodiment, the electrochemically activated water has from 1 to 150 ppm free available chlorine (FAC), preferably from 5 to 100 ppm free available chlorine (FAC). Higher levels are also contemplated.

The efficacy of this disinfectant and the period of time that the activated water holds this disinfectant ability relies on a number of variables including, for example, the purity of the water before it was activated, the amount of charge applied, the period of time it was applied for, the environment it is in, etc. The level of activation can be readily determined by appropriate testing, such as REDOX determination, free available chlorine, microbicidal tests etc. Similarly, the required activation for particular products can be easily determined by conventional testing.

The ECA may have a dual functionality. Once the activated water has been applied to a product or mixed with the other compatible natural or chemical ingredients, it acts as a disinfectant within the product. Once it is in the packaging, it may also disinfect the interior of the pack allowing for a sterile sealed pack/container to be produced. The activated water will act as a preservative to the product for a period of time thereafter. This period of time relies on a number of factors such as the amount of time required by the product given its typical use, the grade and amount of activated water used in the product, the conditions in which it was produced, stored, etc. The required quantity and quality of activated water can be chosen to suit the required product needs. After a period of time the activated water will lose its disinfectant/preservative ability and return to its original or natural state.

Ideally, the electrochemically activated water (ECA) is present at a level of 20 ppm or greater, preferably 50 ppm or greater, more preferably 100 ppm or greater. We have found that levels of approximately 100 ppm are particularly advantageous.

Optionally, the ECA may be present at levels of 200 ppm or greater, preferably greater than 1000 ppm, 1200 ppm, 1500 ppm. Higher levels of ECA may be required if additional agents or formulation ingredients are used or if the material or substrate the formulation is applied to has a high bio-burden and accordingly requires a higher level of ECA.

### Optional Ingredients

To extend the shelf-life of the formulation of the invention and products, it may be desirable in some cases to add a further natural preservative agent.

Such additional ingredients may also be selected from the following an essential oil, such as sweet almond oil, olive oil, lavender oil, aloe vera gel, jojoba oil, peppermint oil, clove oil and/or shea butter.

Additionally, it will be understood that if other agents, such as aloe, rosewater etc., are added, it may be necessary to increase the GSE content to offset potential neutralisation effects of such additional agents.

Further, antimicrobial agents, such as nano silver particles could be added to the product, formulation or packaging material.

In addition, conventional excipients/ingredients may be added. Such excipients will depend on the end form of the formulation i.e. whether it is a liquid, gel or solid and the end use.

### General Applications

According a further aspect of the invention, there is provided a personal care product comprising the natural disinfectant and preservative formulation of the invention.

The personal care product may be selected from wet wipes, creams, lotions, deodorants, formulations, sprays, powders, make-up, shampoo, mousse, paste and/or gels.

For wet wipes, the formulation ideally comprises at least 50 ppm, preferably 100 ppm ECA, and at least 500 ppm, preferably 1000 ppm GSE.

Thus, the formulation of the invention may be used in the disinfection and preservation of personal care products and/or packaging for the personal care product.

The preservative formulation of the invention can be used in many different applications, not just wet wipes. Ideally, for other uses the formulation ideally comprises ECA in the range of 20 ppm to 200 ppm, preferably 50 to 100 ppm, and GSE in the range of 500 to 2500 ppm, preferably 1000 ppm.

For the treatment of skin and hair conditions (with an expected high bio-burden), the use of higher levels of ECA may be contemplated, such as 1000 ppm, 1200 ppm or even 1500 ppm ECA.

For example, the preservative formulation of the invention may be used in oral and periodontal care, such as a toothbrush cleaner or a breath freshener.

In addition, the preservative formulation of the invention may be used in personal care products such as hygiene, cosmetic, baby, oral, skin and hair care products. Ideally, it may be used in shampoo and/or body treatments (e.g. moisturizers, cleansers etc).

We have surprisingly found that a formulation with levels of at least 1000 ppm GSE and at least 200 ppm ECA inhibit the growth of the dandruff causing yeast Malaseezia globosa.

The preservative formulation of the invention may be used to disinfect the packaging for the product and/or in the formulation itself to act as a preservative.

In this way, the preservative formulation of the invention may be used alone or combined with other ingredients, including but not limited to gelling agents, fragrances such as essential oils, and/or conventional ingredients used in shampoos or body treatment products.

It will also be understood that the preparation could also be used for both human and veterinary use, for example for animal treatment products.

According to additional embodiments of the invention, the formulation of the invention may have applications beyond that of personal care products. For example, the formulation may be used in the killing or destroying of micro-organisms in any substrate for preservation or storage, odour control or amending other handling characteristics, including medical instruments and apparatus, dressings and their packaging, and containers for food, drink or pharmaceutical products.

A further embodiment of the invention is directed to a method of disinfecting a medical device and/or packaging for the medical device comprising the use of the formulation of the invention.

Additionally, the formulation may be used in the treatment of water.

A still further embodiment related to the formulation of the invention use in therapy, preferably for use as an antimicrobial agent.

Furthermore, the formulation may have medical applications in the treatment of microbial infections such as wound irrigation and dressing.

Ideally, the formulation of the invention is used in a method of wound irrigation.

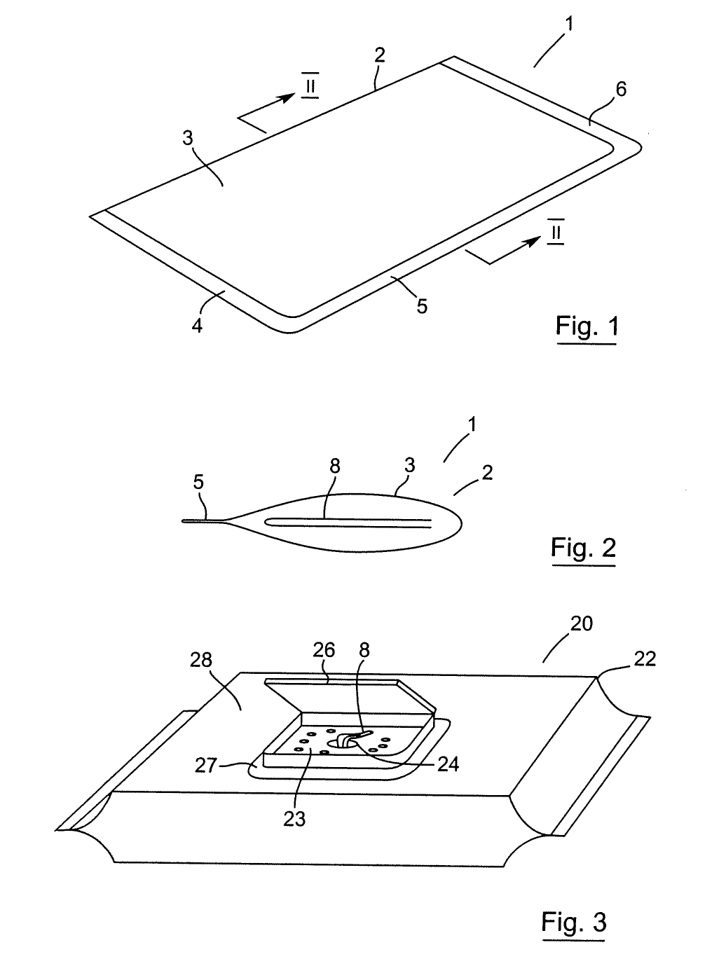

Referring to the drawings and initially to FIGS. 1 and 2 thereof, there is illustrated a wet wipe pack according to the invention indicated generally by the reference numeral 1. The wet wipe pack 1 comprises a sealed outer package 2 of plastics material. The package 2 is formed by folding over a sheet of plastics material to form an envelope 3 with sealed edges 4, 5, 6. A folded wet wipe 8 is retained within the envelope 3. In this case the wet wipe 8 comprises a folded sheet of non-woven material impregnated with mostly activated water or an activated water based formulation.

In use, the activated water maintains a sterile atmosphere within the package 2. A user simply tears open the package 2 and removes the wipe 8 for use as required. After use the wipe 8 is disposed of in the usual way.

Referring now to FIG. 3 there is shown another wet wipe pack 20 which in this case comprises a sealed outer flow-wrap plastics package 22 containing a stack of wipes 8 impregnated with activated water. An outlet of the package 22 is provided with an elastomeric membrane 23 with dispensing opening 24 through which the wipes 8 are removed from the package 22. A cover 26 is mounted across the opening on a support ring 27 fixed to a top wall 28 of the package 22. Said cover 26 can be closed to retain the moisture within the package 22. The elastomeric membrane 23 with dispensing opening 24 also resists moisture loss from the package 22 and migration of bacteria-laden air and other contaminants into the package 22. This elastomeric membrane 23 with dispensing opening 24 (and/or the surrounding area of cover 26 and support ring 27) may also include an anti-microbial agent such as nano silver particles.

It will be appreciated that the invention provides a sealed convenient pack of wipes or pads in which the wipes or pads are simply impregnated with activated water or an activated water based formulation, free from or with minimal amounts of conventional chemical preservatives, which provides an anti-bacterial and preservative function as well as moistening the wipes.

It is envisaged that in some cases it may be desirable to add other agents to the activated water such as cleaning agents, perfumes and the like.

The invention envisages the provision of many types of personal care products or cleaning products as previously mentioned incorporating activated water. Any suitable packaging for such products may also be provided.

### EXAMPLES

### General Materials

Commercially available cotton, viscose or manmade fibre sheets for use as wipes, nonwoven or other
Plastic packaging, whether laminated PA/PE, PP/PE or other construction, typically used for packing wet wipes and other wet products
Commercially available tinfoil packaging
Reverse Osmosis Water used unless otherwise stated
Mains Supply Tap water & Sterile Water
GSE—no significant content of impurities or synthetic biocides (such as benzalkonium chloride etc) and is bactericidal against a panel of gram positive and gram negative bacteria and prevented growth of the yeast Candida albicans and the fungus Aspergillus nidulans at a dilution of 1 in 2000 or 500 ppm in standard suspension tests as per EN 1040 and EN 1275.
Activated water—anolyte and catholyte admixed to yield, pH 6-8, Redox +400 mV-900 mV

### Comparative Example 1

### Bio-Burden on Viscose Interleaved Wipe Matrix, Cotton Wipes and Plastic Packaging

### Materials & Method

Raw materials for the manufacture of wipes were subjected to testing for bioburden according to methods set out in BS EN ISO 11737-1:2006.

Samples of the respective materials (viscose, cotton, plastic packaging) were handled aseptically and were subjected to washing and or swabbing extraction methods. 25 g amounts of viscose or cotton were extracted using sterile distilled water using stomaching for 5 minutes. Plastic packaging was sampled by swab and rinse techniques. The isolation media used was Plate Count Agar and Sabaroud Dextrose Agar.

### Results

The results show that viscose interleaved wipes matrix contained both bacterial and fungal contaminants in varying amounts attached to or embedded in the matrix. Samples of cotton were also shown to contain a variety of micro-organisms, albeit in greater numbers than found in the viscose. These included Bacillus spores which are indicative of soil and which are difficult to destroy. These microbes could accumulate from a number of sources in the production process, raw materials, air, water, transportation, handling etc as reflected by their variety.

| | | |
|---|---|---|
| | Bacterial | Yeasts and moulds |
| Test material | cfu/g | cfu/g |

| | | | |
|---|---|---|---|
| Viscose | batch 1 | 6 | 27 |
| Viscose | batch 1 | 2 | 12 |
| Viscose | batch 2 | 0 | 11 |
| Viscose | batch 2 | 13 | 8 |
| Viscose | batch 3 | 11 | 5 |
| Viscose | batch 3 | 7 | 17 |
| Viscose | batch 3 | 4 | 6 |
| Cotton | batch 1 | 12 | 14 |
| Cotton | batch 1 | 9 | 21 |
| Cotton | batch 2 | 16 | 7 |
| Cotton | batch 2 | 8 | 25 |
| Cotton | batch 3 | 8 | 4 |
| Cotton | batch 3 | 11 | 41 |
| Cotton | batch 3 | 14 | 32 |

The plastic packaging materials which are manufactured by heat extrusion processes are generally sterile at the point of manufacture. The bioburden recorded for these materials, although low numbers, indicated that these are picked up after manufacture. This could be at any point from air, transport contact, handling etc and may be exacerbated by attractive forces of static electricity.

| | Bacterial | Yeasts and moulds |
|---|---|---|
| Test material | cfu/g | cfu/g |
| Plastic lid 1 | 0 | 2 |
| Plastic lid 2 | 0 | 4 |
| Plastic lid 3 | 16 | 1 |
| Pouch 1 | 4 | 0 |
| Pouch 2 | 4 | 3 |
| Pouch 3 | 0 | 3 |

### Conclusions

We have found that both matrix and packaging contain a selection of bacteria and fungi. The distribution of microbes is uneven. Cotton contains a higher bioburden than viscose. All of these factors will contribute to spoilage of wet wipes over time unless: (a) the bioburden is reduced to zero; and/or (b) substantial inhibitors are added.

### Comparative Example 2

### Storage Properties of Minimally Treated Viscose Wipes

### Materials & Method

72 sheet batches of viscose moistened with sterile water and mains supply tap water at a rate of 10 ml per gram were tested. A sterile control pack was prepared by autoclaving moistened viscose wipes in a tinfoil pack. All batches were packed in sterile clear plastic packs under aseptic conditions and were stored at room temperature in the light. The packs were stored in sealed condition.

### Results

The results for visible spoilage are shown below. (the tabulation of some of these charts needs to be adjusted)

| | Day 7 | Day 14 | Day 21 | Day 28 |
|---|---|---|---|---|
| Sterile control | No spoilage | No spoilage | No spoilage | No spoilage |
| Sterile water | No spoilage | Black spots | Intensifying | Intensifying |
| pack 1 | | | | |
| Sterile water | No spoilage | Green spots | No change | No change |
| pack 2 | | | | |
| Sterile water | No spoilage | Grey corner | Grey | Blackening |
| pack 3 | | | throughout | |
| Tap water | No spoilage | Blackening | Blackening | Black/green |
| pack 1 | | | | |
| Tap water | No spoilage | Blackening | Blackening | Blackening |
| pack 2 | | | | |
| Tap water | Grey spot | Blackening | Blackening | Blackening |
| pack 3 | | | | |

The spoilage commenced during the second week of storage in batches prepared using both sterile water and mains tap water. This spoilage occurred mainly around the edges of the wipes. This was as expected in the aerobic zones within the package.

The control pack which had first been sterilised showed no spoilage over the 4 week study and subsequently remained in the same condition for several months until opened. No viable microbes were recovered from the pack at that time.

### Conclusion

This showed that the use of sterile water to moisten the wipes provided conditions for spore outgrowth from the viscose matrix itself. The use of non-sterile mains water caused faster and increased levels of spoilage. The conclusion was that the wipes and moistening agent, if initially sterilised, would remain sterile until opened. An additive or treatment would be necessary to achieve and maintain this state to ensure adequate shelf life in case of pack damage and during normal use after opening.

### Example 3

### Use of GSE Water Used as the Sole Moistening Agent with Viscose Wipes

### Materials & Methods

Batches of wipes were prepared according to comparative example 2 using sterile distilled water and mains tap water but in this case GSE was added to mains tap water at concentrations from 100 ppm to 1500 ppm and the wipes were moistened at 10 ml/g.

### Results

| | Day 7 | Day 14 | Day 21 | Day 28 |
|---|---|---|---|---|
| Sterile control | No spoilage | No spoilage | No spoilage | No spoilage |
| GSE 200 ppm | No spoilage | Grey spots | No change | Intensifying |
| GSE 500 ppm | No spoilage | No spoilage | Greying at | No change |
| | | | edges | |
| GSE 1000 ppm | No spoilage | No spoilage | No spoilage | 1 Grey spot |
| GSE 1500 ppm | No spoilage | No spoilage | No spoilage | No spoilage |

These results show that GSE at 1000 ppm or above protected the wipes from spoilage for at least three weeks. One spot appeared on the 1000 ppm GSE wipes at 4 weeks but failed to progress further. This suggests that the GSE could simply be restricting growth as opposed to killing off the bacteria completely.

No bacteria were cultured from 1000 or 1500 ppm packs when opened. These packs were kept sealed and unopened therefore we would not be confident that GSE alone at these concentrations would protect the pack in open conditions, or where the bacteria loadings are higher than those in this test, for example if the dry wipe bundles were not as clean as this batch was to start with.

### Example 4

### Use of Electrochemically Activated Water Alone

### Materials & Method

Batches of wipes were prepared as per comparative example 2 and exposed to 20 ppm electrochemically activated water for 10 and 20 minutes respectively.

### Results

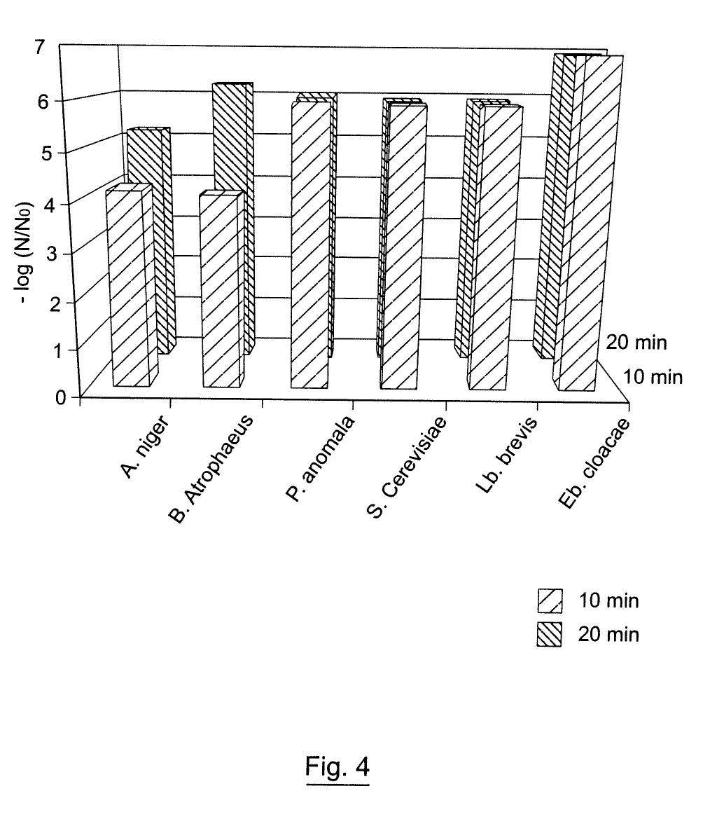

As shown in FIG. 4, using 20 ppm Electrochemically activated water, a 6 or 7 log reduction was achieved for bacterial and yeast species within 10 minutes. Only in the cases of a sporing bacillus and a fungus did it take longer to improve on the kill rate. This shows electrochemically activated water is effective against both fungi and bacteria, including spores.

### Example 5

### Electrochemically Activated Water Alone Used As the Sole Moistening Agent with Viscose Wipes

### Materials & Methods

Batches of wipes were prepared as per comparative example 2 using sterile distilled water and mains tap water but in this case electrochemically activated water was added to mains tap water at concentrations from 1 ppm to 200 ppm and the wipes were moistened at 10 ml/g.

### Results

| | Day 7 | Day 14 | Day 21 | Day 28 |
|---|---|---|---|---|
| Sterile control | No spoilage | No spoilage | No spoilage | No spoilage |
| Electrochemically | No spoilage | Grey spots | Intensifying | Intensifying |
| activated water 1 ppm | | | | |
| Electrochemically | No spoilage | No spoilage | Greying at edges | No change |
| activated water 5 ppm | | | | |
| Electrochemically | No spoilage | No spoilage | Grey spots | Grey spots |
| activated water 10 ppm | | | | |
| Electrochemically | No spoilage | No spoilage | Slight green spot | Black/green |
| activated water 20 ppm | | | | |
| Electrochemically | No spoilage | No spoilage | Grey spots | Grey spots |
| activated water 50 ppm | | | | |
| Electrochemically | No spoilage | No spoilage | Grey spots | No change |
| activated water 100 ppm | | | | |
| Electrochemically | No spoilage | No spoilage | No spoilage | No spoilage |
| activated water 150 ppm | | | | |
| Electrochemically | No spoilage | No spoilage | No spoilage | No spoilage |
| activated water 200 ppm | | | | |

These results show that electrochemically activated water alone could prevent spoilage over the 4 week study when present at 150 ppm or above in sealed packs. At concentrations below that, it appeared that it was able to delay spoilage for up to three weeks and restrict the degree of spoilage to some extent between 5 and 100 ppm. No bacteria were isolated from the 1, 5, 10 or 20 ppm packs when opened after 4 weeks, indicating only fungal survival.

Although there was no spoilage in the wipes or no bacteria surviving at the higher PPM in these sealed packs, we found that there was also no remaining residual effect once these packs were opened. Thus, the ECA is only effective in closed/sealed conditions.

### Example 6

### Reverse Osmosis (RO) Water, Electrochemically Activated Water Plus Cognis Additive Compared with RO Water, Electrochemically Activated Water Plus GSE

### Materials & Methods

Comparative experiments using electrochemically activated water plus Cognis® additive (an off the shelf preservative/cleansing formulation) or GSE at one of two concentrations were carried out.

72 sheet batches of viscose moistened with reverse osmosis water plus ECA water and GSE or Cognis® additive at a rate of 10 ml per gram. A sterile control pack was prepared by autoclaving moistened viscose wipes in a tinfoil pack. All batches were packed in sterile clear plastic packs under aseptic conditions and were stored at room temperature in the light. The packs were stored in sealed condition.

### Results

| | Day 30 | Day 50 | Day 72 |
|---|---|---|---|
| Sterile control | No spoilage | No spoilage | No spoilage |
| Electrochemically activated | No spoilage | Green spot | Green spot |
| water 20 to 100 ppm + Cognis | | | |
| Electrochemically activated | No spoilage | No spoilage | No spoilage |
| water150 to 200 ppm + Cognis | | | (in closed conditions) |
| Electrochemically activated | No spoilage | No spoilage | Grey spots |
| water 20 ppm + GSE 500 | | | |
| Electrochemically activated | No spoilage | No spoilage | Green spot |
| water 50 ppm + GSE 500 | | | |
| Electrochemically activated | No spoilage | No spoilage | No spoilage |
| water 100 ppm + GSE 500 | | | |
| Electrochemically activated | No spoilage | No spoilage | No spoilage |
| water 150 ppm + GSE 500 | | | |
| Electrochemically activated | No spoilage | No spoilage | No spoilage |
| water 200 ppm + GSE 500 | | | |
| Electrochemically activated | No spoilage | No spoilage | No spoilage |
| water 20 ppm + GSE 1000 | | | |
| Electrochemically activated | No spoilage | No spoilage | No spoilage |
| water 50 ppm + GSE 1000 | | | |
| Electrochemically activated | No spoilage | No spoilage | No spoilage |
| water 100 ppm + GSE 1000 | | | |
| Electrochemically activated | No spoilage | No spoilage | No spoilage |
| water 150 ppm + GSE 1000 | | | |
| Electrochemically activated | No spoilage | No spoilage | No spoilage |
| water 200 ppm + GSE 1000 | | | |
| Electrochemically activated | No spoilage | No spoilage | No spoilage |
| water 20 ppm + GSE 1500 | | | |
| Electrochemically activated | No spoilage | No spoilage | No spoilage |
| water 50 ppm + GSE 1500 | | | |
| Electrochemically activated | No spoilage | No spoilage | No spoilage |
| water 100 ppm + GSE 1500 | | | |
| Electrochemically activated | No spoilage | No spoilage | No spoilage |
| water 150 ppm + GSE 1500 | | | |
| Electrochemically activated | No spoilage | No spoilage | No spoilage |
| water 200 ppm + GSE 1500 | | | |

These tests showed that a combination of RO water, Electrochemically activated water at 50 ppm/100 pppm or above and GSE at 500 ppm or above, provided spoilage prevention for over six weeks and was comparable to the performance of conventional formulations that contain conventional bacteriosides or preservatives such as Nipagard® BXP. No bacteria were recovered from any of the packs when opened at 72 days.

This example shows a synergistic effect between electrochemically activated water and GSE in preventing spoilage beyond their individual effects alone. For example, electrochemically activated water alone at 100 ppm or GSE 500 ppm alone delayed spoilage for only two weeks but together delayed it for over 6 weeks.

### General Conclusions

These examples show that both the packaging materials and the wipes matrix contained a range of microbes (mainly fungi). When packed and moisture was added, spoilage occurred within the packs within as little as two weeks when stored at room temperature, without being opened.

This would not occur if a chemical biocide such as Nipagard® BPX had been added to the moisturising liquid.

We have now shown that electrochemically activated water and GSE are microbicidal. Minimal concentrations were established which could prevent spoilage when used alone or in combination. A combination of both resulted in a synergistic interaction and greatly enhanced preservative function (refer to Comparative Examples 3 to 5 which showed the use of ECA alone or GSE alone).

### Example 7

### Preparation of Prototype Novel Wipes Packs for Shelf-Life and Antimicrobial Effect Testing

Batches of wipes packs (108 wipes per pack) were prepared by using reverse osmosis water, disinfecting using ECA at 100 or 200 ppm and adding 1000 ppm or 2000 ppm GSE for residual effect. The packs were held at 20° C. for over eight months.

Representative packs of each combination were held as follows:
in the original sealed condition for the full period before opening and testing
opened initially, 20 wipes removed, one left protruding and stored for the full period before examining and testing
as for both cases above but tested every two weeks initially and then every four weeks

A small series of ziploc pouches containing cotton pads moistened with the same combinations of ECA and GSE were also prepared and tested at intervals over the same eight months.

### Test Methods

### Sterility/Bioburden Testing of Wipes

Single wipes were drawn from the pack aseptically and rinsed in 10 ml sterile water. A 1 ml sample from this rinse was used to prepare a pour plate of Tryptone Soya Agar to determine the Total Viable Count (TVC) incubated at 30° C. for 3 days.

### Challenge Testing

Four test discs were punched from each sample wipe and were applied to lawns of challenge microbial species according to the standard test method ISO 20645:2004. This method prepares an overlay layer of microbes embedded in agar on top of which the test discs are placed. Any antimicrobial activity in the test material diffuses out into the agar and causes inhibition of microbial growth around and under the disc. These zones are measured by micrometer gauge and indicate the residual antimicrobial activity in the wipe at the time of testing.

Microbial strains used in these tests were ATCC culture collection strains or WT (wild type) of gram positive and gram negative bacteria and yeasts.

Control wipes were untreated, autoclaved and moistened with sterile distilled water for use in sterility tests and challenge tests.

### Results Summary

### Sterility/Bioburden Testing of Wipes

All wipes remaining in sealed packs for the eight month period were sterile when tested
All wipes from opened packs except the ECA100 ppm-GSE500 ppm were sterile.
Only a few of the top wipes from several of the ECA100 ppm-GSE500 ppm packs were positive
Cotton pads in the Ziploc pouches showed the same sterile results for the higher concentrations, however, for the ECA100 ppm-GSE500 ppm, mould growth was quite heavy after 3 months.

### Conclusion

It appears that the ECA 100 ppm and GSE 500 ppm and above combination or higher levels, provides a shelf-life of at least eight months for sealed packs but to provide a safety margin, ECA200 ppm and GSE1000 ppm or higher should be used to contend with open packs.

We have also established that ECA200 ppm and GSE 1000 ppm and above is preferable for use with cotton wool, or for example, if other ingredients such as moisturisers etc are added. These conclusions are applicable to the cotton matrix used only.

If other matrices are used such as spunlace, ECA100 ppm and GSE1000 ppm may be used.

### Example 8

### Further Shelf-Life Trials of Wipes Comprising Electrochemically Activated Water and GSE

### Materials & Methods

Using combinations of 100 ppm and 200 ppm electrochemically activated water and 500 and 1000 ppm GSE shelf life over a period of 2 years in both open and shut conditions was tested.

### Results

All wipes in 100 ppm Electrochemically activated water-500 ppm GSE packs which remained closed until testing remained sterile for 2 years. Pack A and pack B are duplicates. All are cotton matrices.

Some of the 100 ppm Electrochemically activated water-500 ppm GSE packs which were left open developed spoilage in the top few wipes. All of the 200 ppm Electrochemically activated water-1000 ppm GSE packs whether in open or closed condition remained sterile for 2 years.

| | | | | | | | 87 weeks | |
|---|---|---|---|---|---|---|---|---|
| Electrochemically | | | | | | | storage | |
| activated water | | | | | | | 16/2/09 | |
| 200 ppm | Open | | Open | | Closed | | Closed | |
| GSE 1000 ppm | Pack A Wipe 1 | | Pack A Wipe 2 | | Pack B Wipe 1 | | Pack B Wipe 2 | |
| TVC @30° C. | No Growth | | No Growth | | No Growth | | No Growth | |
| Challenge | | | | | | | | |
| S. aureus 6538 | 1.1 mm | 0 | <1.0 mm | 0 | 1.0 mm | 0 | 1.5 mm | 0 |
| S. aureus WT | 1.0 mm | 0 | <1.0 mm | 0 | 1.1 mm | 0 | 1.5 mm | 0 |
| E. coli 11229 | 1.2 mm | 0 | 1.2 mm | 0 | 1.2 mm | 0 | 1.7 mm | 0 |
| E. coli WT | 1.1 mm | 0 | 1.2 mm | 0 | 1.0 mm | 0 | 1.3 mm | 0 |
| C. albicans 36232 | 1.1 mm | 0 | 1.1 mm | 0 | 1.4 mm | 0 | 1.5 mm | 0 |
| C. albicans WT | 0.2 mm | 0 | 0.0 mm | 0 | 0.4 mm | 0 | 0.2 mm | 0 |
| CONTROL | 0 | + | | | | | | |

### Conclusion

The above table shows that the 200 ppm Electrochemically activated water to 1000 ppm GSE treated packs are stable over a period of years in a closed state and are also resistant to spoilage under normal use in opened state.

The cotton pads in pouches had a higher bioburden of moulds initially and certainly require the higher levels of additives to maintain shelf-life and sterility. Thus, it is reasonable to expect that 50 ppm/100 ppm ECA-500 ppm GSE will be applicable in other situations.

### Example 9

### Additional of Additives/Functional Enhancers

### Materials & Method

A number of potential additives which could enhance the functionality of wipes or solutions to be used with wipes were tested for computability and inhibition of microbicidal effect with 200 ppm Electrochemically activated water-1000 ppm GSE, 200 ppm electro-chemically activated water-1500 ppm GSE and 200 ppm electrochemically activated water-2000 ppm GSE.

These additives include sweet almond oil, olive oil, lavender oil, aloe vera gel, jojoba oil, peppermint oil, clove oil and shea butter. All additives were tested at approximately 1% final volume. All of the additives were miscible with the solutions at 37° C.

Inhibition tests were carried out using disk diffusion on a lawn of wild type S. aureus, 48 hours incubation at 37° C. Figures are mm inhibition zone.

### Results

| | Electrochemically | Electrochemically | Electrochemically |
|---|---|---|---|
| | activated water/ | activated water/ | activated water/ |
| | GSE | GSE | GSE |
| | 200/1000 | 200/1500 | 200/2000 |
| Sweet almond | 1.9 | 3.0 | 4.2 |
| oil | | | |
| Olive oil | 2.0 | 2.2 | 2.6 |
| Lavender oil | 2.6 | 3.0 | 4.1 |
| Aloe vera gel | 2.9 | 3.9 | 4.0 |
| Jojoba oil | 3.7 | 4.9 | 4.8 |
| Peppermint oil | 4.2 | 5.8 | 6.7 |
| Clove oil | 4.0 | 6.0 | 8.1 |
| Shea butter | 3.3 | 4.1 | 2.5 |
| No additive | 5.6 | 8.0 | 9.2 |

It is concluded that some of the additives have microbicidal activity in their own right e.g peppermint and clove oils. All have impact on the inhibitory activity of the Electrochemically activated water/GSE solutions to some extent, however, the 1500 ppm and 2000 ppm GSE appears to cope with this. It is probable that the higher GSE concentration would need to be used with these additives for safeguarding shelf-life of wipes.

It will be understood that the preservative formulation can be used in many different applications. Some examples of these applications follow:

### Example 10

### Toothbrush Hygiene

### Method

A formulation comprising 100 ppm ECA and 1000 ppm GSE dispersed in RO water was made. This formulation was used to fill a travel toothbrush cap or lid in order to immerse the toothbrush head for a period of 2 minutes or longer.

10 toothbrushes were sampled for microbial counts after 1 weeks normal usage and overnight storage.

Each toothbrush was vortexed in 10 ml sterile saline and this was then plated out on blood agar (0.1 ml spread plates) and incubated at 37° C. for 48 hours. Estimated bacterial counts were measured using conventional means.

### Results and Conclusions

The estimated bacterial counts per toothbrush averaged 7.3×10⁴ cfu. The same toothbrushes were returned to their owners and used for another week. Pairs of brushes were then stored for different periods (2 minutes, 10 minutes, 20 minutes, overnight) in the solution (ECA/GSE only) and 2 in sterile water as a control.

Sampling was done as previous examples.

| | |
|---|---|
| The control samples averaged | 1.5 × 10⁵ |
| 2 minute | 1.1 × 10¹ |
| 10 minute | 2.0 × 10¹ |
| 20 minute | 2.2 × 10¹ |
| Overnight. | 1.6 × 10¹ |

All treated brushes showed greater than 1000-fold reduction in viable bacteria whether 2 minutes or overnight. In microbiological terms the numbers of residual bacteria were negligible.

### Conclusion

We found that rinsing the toothbrush after use and immersing the head in the formulation cleaned and freshened the toothbrush and prevented microbial growth.

Other additives may be added to the formulation, such as 2% (total volume) clove oil or peppermint oil or other flavouring agent depending on preference or a foodsafe gelling agent (e.g. carageenan) for travel purposes etc.

It will be understood that the formulation may comprise from 20 ppm to 200 ppm, preferably from 50 to 100 ppm, ECA and from 500 to 2500 ppm GSE. The exact level of ECA and GSE will depend on the bacterial load/bio-burden of the substrate or material it is being applied to.

### Example 11

### Periodontal Hygiene and Breath Freshener

A mix of ECA/GSE as per Example 10 but using 1200 ppm ECA stock solution to minimise the RO water volume was prepared.

The further mix with 60% (Vol) Sweet almond oil, ECA/GSE mix (20% vol), clove oil, peppermint, arrowmint, Cinnamon or other flavoured or aromatic oil 5% each (Vol) was made. The proportion of sweet almond oil may be increased to decrease the freshness tang and the oils reduced accordingly.

This mix is typically dispensed by a small pump spray bottle (10 ml or greater). One spray onto a soft toothbrush and applied to the periodontal areas will provide cleansing action due to the emulsion and the oil-based components will preferentially adhere to the teeth and gums. Even with salivary washing, the freshening effects will last for several hours unlike many aqueous or alcohol-based mouthwashes. The antimicrobial effects of ECA/GSE are already documented. Clove oil is a known microbicide used for dental purposes as are a range of other essential oils.

The spray can also be used directly into the mouth.

### Example 12

### Hygiene Shampoo or Long Shelf-Life Perishable Specialty Shampoos

A mix of ECA/GSE as per example 10 was prepared initially. A shampoo mix was prepared containing the ECA/GSE, typically containing:

| | |
|---|---|
| ECA/GSE | 10% vol |
| Texapon ® EVR | 40% |
| Degassed Dark stout | 48-49% |
| Fragrance | 1-2% |
| | (e.g. YlangYlang, jasmine, lavender essential oils) |

(TexaponEVR is Cognis ® product consisting of Sodium Lauryl Sulfate, Sodium Laureth, Sulfate Lauramide MIPA, Cocamide MEA, GlycolDistearate and Laureth-10.)

The above formulation is for a concentrated golden pearlescent shampoo with a very rich lather which leaves hair shining. However, there is a rich protein content which might ordinarily spoil or give rise to unwanted odor.

### RESULTS AND CONCLUSIONS

Stability tests have shown 5 years storage without failure when the ECA/GSE mix is added.

Six out of twenty samples without additive spoiled within 20 months by generating gas and in one case rupturing the plastic container.

In separate tests the GSE and ECA have been shown to inhibit the growth of the dandruff causing Malaseezia globosa at 1000 ppm and 200 ppm respectively. This is the yeast most closely associated with dandruff causation.

A range of natural antimicrobials e.g. tea tree oil etc at 1% or zinc pyrithrione 0.2% could also be added for dandruff control/hygiene.

The invention is not limited to the embodiments hereinbefore described which may be varied in both construction and detail.

1. A natural disinfectant and preservative formulation comprising electrochemically activated water and grapefruit seed extract.

2. A formulation according to claim 1 dispersed in a diluent such as sterilised, filtered or purified water, preferably reverse osmosis water.

3. A formulation according to claim 1 comprising at least 20 ppm electrochemically activated water and at least 500 ppm grapefruit seed extract.

4. A formulation according to claim 3 comprising at least 50 ppm electrochemically activated water.

5. A formulation according to claim 1 wherein the combination of the electrochemically activated water and the grapefruit seed extract provides for an efficacy greater than the efficacy of either administered alone.

6. A formulation according to claim 1 in a sealed outer package, preferably a sealed container.

7. A formulation according to claim 1 wherein the electrochemically activated water is present at a level of 100 ppm, optionally 100 ppm or greater.

8. A formulation according to claim 1 wherein the electrochemically activated water has a pH from 2 to 9, preferably 3 to 8.5, more preferably from 6.5 to 7.5.

9. A formulation according to claim 1 wherein the electrochemically activated water has a redox potential of greater than 400 mV, preferably greater than 600 mV.

10. A formulation according to claim 1 wherein the electrochemically activated water comprises greater than 50%, preferably greater than 75% anolyte.

11. A formulation according to claim 1 wherein the grapefruit seed extract is present at a level of 1000 ppm, optionally 1000 ppm or greater.

12. A formulation according to claim 1 wherein the grapefruit seed extract comprises less than 2%, preferably less than 0.1%, v/v of impurities including quaternary ammonium compounds including benzalkonium chloride, triclosan, pesticide/biocide derived contaminants and/or allergans.

13. A formulation according to claim 1 wherein the grapefruit seed extract is anti-microbial and bactericidal against gram-positive and gram-negative bacteria and prevents growth of Candida albicans and Aspergillus nidulans.

14. A formulation according to claim 1 wherein the grapefruit seed extract comprises approximately
40 to 50%, ideally 45% v/v dextrose free grapefruit seed extract;
30 to 40%, ideally 34% v/v glycerine; and
15 to 25%, ideally 18% v/v vitamin C (ascorbic acid).

15. (canceled)

16. A natural disinfectant and preservative formulation according to claim 1 in the form of a solid, liquid or gel or combined with or applied to a solid, liquid or gel substrate.

17. A personal care product comprising the natural disinfectant and preservative formulation according to claim 1.

18. A personal care product according to claim 17 wherein the personal care product is selected from wet wipes, creams, lotions, deodorants, formulations, sprays, powders, make-up, shampoo, mousse, paste and/or gels

19. A wet wipe comprising a substrate impregnated with natural disinfectant and preservative formulation comprising at least 50 ppm, preferably 100 ppm, electrochemically activated water and at least 500 ppm, preferably 1000 ppm, grapefruit seed extract.

20. A wet wipe pack comprising a sealed outer package containing one or more wet wipes, wherein each of said wet wipes comprising a substrate impregnated with the formulation of claim 1.

21. (canceled)

22. A method of disinfecting and preserving a personal care product and/or packaging for the personal care product comprising applying the formulation according to claim 1 to the personal care product and/or packaging for the personal care product.

23. (canceled)

24. (canceled)

25. (canceled)
